(19) United States
(12) Patent Application Publication
(10) Pub. No.: US 2025/0115864 A1
(43) Pub. Date: Apr. 10, 2025

(54) A FERMENTATION MEDIUM COMPRISING SULPHUR

(71) Applicant: Evonik Operations GmbH, Essen (DE)

(72) Inventors: Thomas HAAS, Münster (DE); Christian RICHTER, Münster (DE); Martin DEMLER, Dorsten (DE); Simon BECK, Münster (DE)

(73) Assignee: Evonik Operations GmbH, Essen (DE)

(21) Appl. No.: 18/695,470

(22) PCT Filed: Sep. 28, 2022
(86) PCT No.: PCT/EP2022/076946

§ 371 Date: Mar. 26, 2024

(30) Foreign Application Priority Data
Sep. 30, 2021 (EP) 21200133.3

(51) Int. Cl. C12N 1/20; C12P 7/06; C12P 7/40

(52) U.S. Cl. CPC C12N 1/20; C12P 7/06; C12P 7/40; C12N 2500/20; C12N 2500/24; C12N 2500/44

(57) ABSTRACT

The present invention relates to an aqueous fermentation medium comprising sulphur in the form of at least one thiocarboxylate, wherein the thiocarboxylate has a chemical structure of Formula I and R═H, alkyl, COOH, COSH, and wherein the alkyl groups may also contain OH, COSH and/or COOH and wherein the concentration of the thiocarboxylate is 2 to 20 mg/L in the fermentation medium.

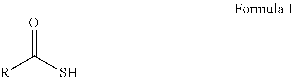

### FIELD OF THE INVENTION

The present invention relates to a fermentation medium and use thereof for producing organic compounds from a carbon source in the presence of hydrogen and anerobic organisms. In particular, the fermentation medium comprises sulphur in the form of thiocarboxylates that enables the anerobic organisms to produce organic products from the carbon sources available.

### BACKGROUND OF THE INVENTION

In the fermentation process, anaerobic organisms convert CO₂, CO, H₂ and/or other carbohydrates to a variety of organic products such as lactic acid, acetic acid, ethanol and the like. There are numerous conventional methods that exist for sustaining a microorganism culture that is capable of producing a variety if useful organic products during fermentation. However, these methods suffer from numerous inefficiencies. Several of these microorganisms are delicate by nature and susceptible to slight changes in the surrounding conditions in the cultural medium. This reduces the efficiency of production of useful organic products from a suitable carbon source. Microorganisms used in these fermentation processes require besides water as the reaction media also certain elements and vitamins to live, grow and reproduce. Nutrients and micronutrients and the particular supply of these nutrients can have profound effects on the growth and sustainability of the microorganisms.

One of the crucial nutrients in the fermentation medium is a source of sulphur. Besides sulphur, iron, nickel and cobalt are also essential in the fermentation medium. Usually, sulphur is in the form of a chemically reduced state like sulfide. In particular, sulphur is often provided in the medium as H₂S, Na₂S, or alternatively as cysteine. If H₂S or Na₂S are used, the solubility products of the resulting Fe, Ni or Co salts are often exceeded, and these salts will end up precipitating resulting in very negative consequences for pumps and valves used in the fermentation. Also, hydrogen sulfide is also toxic and thus requires special handling and is particularly dangerous in its pure form. Supplying sulphur in the form of a sulfide salt such as sodium sulfide still results in a hydrogen sulfide concentration in the fermenter that may decrease over time due to evaporation. Hydrogen sulfide may also become highly volatile under the conditions that may be desired for fermentation thereby exacerbating its use as a sulphur source. Moreover, hydrogen sulfide has a limited solubility in the fermentation medium. For all these reasons and more, sulfide is not the best source of sulphur for fermentation to be efficiently carried out by cells.

The alternative sulphur source-cysteine is very often digested by the microorganisms themselves thus leading to a large quantity of cysteine needed for each fermentation process. In particular, cysteine is oxidised to the dimer cysteine. This leads to very high cysteine costs.

The reduced form of these sulphur compounds is considered to be more substantially bioavailable as a sulphur source for use by a microbial culture than the oxidised form. As such, when a sulphur source is used to lower the Oxidation-reduction potential (ORP) of a fermentation reaction, the actual concentration of sulphur available to the microbial culture will decrease.

WO 2013/147621 discloses a fermentation method of producing alcohols where sulphur is added into the fermentation medium in the form of sulphurous acid (H₂SO₃), SO₂, Na₂S₂O₄, Na₂S, NaHS, cysteine, NH₄HSO₃ or (NH₄)₂SO₃. However, for the sulphur to be present in the medium effectively, CO must also be present. Accordingly, this limits the source of carbon that can be used as a substrate for production of organic compounds.

There is thus a need in the art to provide a sulphur source in a fermentation medium that is not only not too expensive but that can also be efficiently used by the microorganisms and that also supports the enzymatic processes occurring in the microbial culture. The added sulphur source must also be in a bioavailable form and in sufficient supply to avoid inhibiting the growth or production of the organic product by the microorganism.

### DESCRIPTION OF THE INVENTION

The present invention attempts to solve the problems above by providing a fermentation medium comprising sulphur in the form of at least one thiocarboxylate. In particular, the thiocarboxylate has a chemical structure of Formula I:

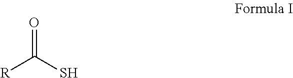

wherein R═H, alkyl, aryl, COOH, COSH, and wherein the alkyl and aryl groups may also contain OH, COSH and/or COOH. More in particular, R═H, alkyl, COOH, COSH, and wherein the groups may also contain OH, COSH and/or COOH.

The use of a thiocarboxylate according to any aspect of the present invention, allows for sulphur to be in a bioavailable form for use by cells in the fermentation medium in the fermentation process for production of organic compounds without oxidising or digesting the thiocarboxylate. There is thus no need to regularly replenish the thiocarboxylate in the medium.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (e.g., isopropyl, tert-butyl, isobutyl, etc.), cycloalkyl (alicyclic) groups (e.g., cyclopropyl, cyclopentyl, cyclohexyl, cycloheptyl, cyclooctyl), alkyl substituted cycloalkyl groups, and cycloalkyl substituted alkyl groups. The term “alkyl” further includes alkyl groups, which can further include oxygen, nitrogen, sulphur or phosphorous atoms replacing one or more carbons of the hydrocarbon backbone. In particular, the alkyl group of Formula I contains 1 to 6 carbon atoms. The term alkyl includes both “unsubstituted alkyls” and “substituted alkyls”, the latter of which refers to alkyl moieties having substituents replacing a hydrogen on one or more carbons of the hydrocarbon backbone. The alkyl group may also contain OH, COSH and/or COOH groups.

The term “aryl” includes groups, including 5- and 6-membered single-ring aromatic groups that may include from zero to four heteroatoms, for example, benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxophenyl, quinoline, isoquinoline, naphthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aryl group may also contain OH, COSH and/or COOH groups.

In particular, the thiocarboxylate may be selected from the group consisting of thioacetate, thioformate, thiobutyrate, thiolactate, thiopropionate, thiohexanoate, thiooctanoate, thiodecanoate, thiododecanoate, thiobenzoate and thiocitrate salt. More in particular, the thiocarboxylate may be selected from the group consisting of thioacetate, thioformate, thiobutyrate, thiolactate, thiopropionate, thiohexanoate, thiooctanoate, thiodecanoate, thiododecanoate, thiobenzoate and thiocitrate salt. Even more in particular, the thiocarboxylate may be selected from the group consisting of thioacetate, thiobutyrate and thiocitrate. Especially more in particular, the thiocarboxylate may be selected from the group consisting of potassium thioacetate, sodium thioacetate, calcium thioacetate, potassium thiobutyrate, sodium thiobutyrate, calcium thiobutyrate, potassium thiocitrate, sodium thiocitrate, calcium thiocitrate and the like.

Thiocarboxylate provides a crucial element in a fermentation medium, sulphur, in high concentrations without precipitations and avoiding digestion of the sulphur. Thus, saving costs and making the fermentation process more efficient.

The concentration of sulphur in the fermentation medium may be about 1 to 100 mg/L. In particular, the concentration of the sulphur may be 1 to 95, 1 to 90, 1 to 85, 1 to 80, 1 to 75, 1 to 70, 1 to 65, 1 to 60, 1 to 55, 1 to 50, 1 to 45, 1 to 40, 1 to 35, 1 to 30, 1 to 25, 1 to 20, 1 to 15, 1 to 10 mg/L in the fermentation medium. More in particular, the concentration of the sulphur may be about, 1, 2, 3, 4, 5, 6, 7, 8, 9 or 10 mg/L in the fermentation medium. The sulphur in the fermentation medium is introduced using thiocarboxylate. In particular, the concentration of thiocarboxylate in the fermentation medium may be 0.02 to 0.3 mmol/L. More in particular, the concentration of thiocarboxylate in the fermentation medium may be 0.02 to 0.25, 0.02 to 0.2, 0.02 to 0.15, 0.02 to 0.10, 0.02 to 0.05, 0.05 to 0.25, 0.05 to 0.2, 0.05 to 0.15, 0.05 to 0.10, 0.07 to 0.25, 0.07 to 0.2, 0.07 to 0.15, 0.07 to 0.10, 0.10 to 0.25, 0.10 to 0.2, 0.10 to 0.15, 0.15 to 0.25, 0.15 to 0.2, 0.20 to 0.25,

In one example, the thiocarboxylate may be potassium thioacetate and the concentration of thioacetate molecules in the fermentation medium may be 2 to 20 mg/L. In particular, and the concentration of thioacetate molecules in the fermentation medium may be 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 5 to 20, 5 to 18, 5 to 17, 5 to 16, 5 to 15, 5 to 14, 5 to 13, 5 to 12, 5 to 11, 5 to 10, 10 to 20, 10 to 19, 10 to 18, 10 to 17, 10 to 16, 10 to 15 or 15 to 20 mg/L. In one example, the thiocarboxylate may be potassium thioacetate and the concentration of thioacetate molecules in the fermentation medium may be 1 to 14, 1 to 13, 1 to 12, 1 to 11, 1 to 10, 1 to 9, 1 to 8, 1 to 7, 1 to 6 or 1 to 5 mg/L. The concentration of thioacetate molecules in the fermentation medium may be 2 to 15, 2 to 12, 2 to 10 mg/L. More in particular, the concentration of thioacetate molecules in the fermentation medium may be about 1, 2, 3, 4, 5, 6, 7, 8, 9, 10, 11, 12, 13, 14, 15, 16, 17, 18, 19 or 20 mg/L.

The term ‘about’ as used herein refers to a variation within 20 percent. In particular, the term “about” as used herein refers to +/−20%, more in particular, +/−10%, even more in particular, +/−5% of a given measurement or value.

The fermentation medium may comprise other elements at specific concentrations for the cells to carry about fermentation efficiently. In particular, the other elements may be selected from the group consisting of aluminium, boron, calcium, cobalt, magnesium, iron, manganese, molybdenum, potassium, nickel, selenium, tungsten, and zinc. More in particular, the other elements may be selected from the group consisting of iron, nickel and/or cobalt. In one example, the fermentation medium may comprise a thiocarboxylate according to any aspect of the present invention and iron. In another example, the fermentation medium may comprise a thiocarboxylate according to any aspect of the present invention and nickel. In yet another example, the fermentation medium may comprise a thiocarboxylate according to any aspect of the present invention and cobalt. In one example, the fermentation medium may comprise a thiocarboxylate according to any aspect of the present invention, iron and nickel. In a further example, the fermentation medium may comprise a thiocarboxylate according to any aspect of the present invention, iron and cobalt. In one example, the fermentation medium may comprise a thiocarboxylate according to any aspect of the present invention, nickel and cobalt. In one other example, the fermentation medium may comprise a thiocarboxylate according to any aspect of the present invention, iron, nickel and cobalt.

The fermentation medium may comprise 2 mg/L to 5 mg/L of iron. More in particular, the fermentation medium may comprise 3-5 mg/L of iron. Even more in particular, the fermentation medium according to any aspect of the present invention may comprise about 3, 4.5, or 5 mg/L of iron.

In one example, the fermentation medium may further comprise cobalt. The fermentation medium may comprise 480 μg/L to 500 μg/L of cobalt. More in particular, the fermentation medium may comprise 490-500 μg/L or cobalt. Even more in particular, the fermentation medium according to any aspect of the present invention may comprise about 495, 495.5 or 496 μg/L of cobalt.

In one example, the fermentation medium may further comprise nickel. The fermentation medium may comprise 40 μg/L to 55 μg/L of nickel. More in particular, the fermentation medium may comprise 45-50 μg/L of nickel. Even more in particular, the fermentation medium according to any aspect of the present invention may comprise about 49, 49.5 or 50 μg/L of nickel.

According to another aspect of the present invention, there is provided a method of producing at least one organic compound from a carbon source, the method comprising:
contacting at least one anaerobic cell to the carbon source in a fermentation medium,
   wherein the fermentation medium comprises sulphur in the form of at least one thiocarboxylate and wherein the thiocarboxylate has a chemical structure of Formula I:

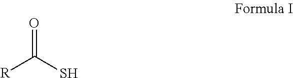

and R═H, alkyl, aryl, COOH, COSH, and wherein the alkyl and aryl groups may also contain OH, COSH and/or COOH. In particular, R═H, alkyl, COOH, COSH, and wherein the alkyl groups May also contain OH, COSH and/or COOH.

This method maintains and/or increases production rates of one or more organic products produced the by anaerobic cell in the fermentation medium. The fermentation efficiency of the anaerobic cell culture may also be improved using thiocarboxylate as the alternative sulphur source in the fermentation medium.

Anaerobic organisms may include carboxydotrophic, photosynthetic, methanogenic and acetogenic organisms. In some examples, the anaerobic bacteria are selected from bacteria of the genus Actinomyces, Bacteroides, Clostridium, Fusobacterium, Peptostreptococcus, Porphyromonas, Prevotella, Propionibacterium, or Veillonella. In one example, the anaerobic bacteria may be an acetogenic cell.

The term “acetogenic cell” or ‘acetogenic bacteria as used herein refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon 40 monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria may be selected from the group consisting of Acetoanaerobium sp., Acetonema sp., Acetobacterium sp., Alkalibaculum sp., Archaeoglobus sp., Blautia sp., Butyribacterium sp., Clostridium sp., Desulfotomaculum sp., Eubacterium sp., Methanosarcina sp., Moorella sp., Oxobacter sp., Sporomusa sp., Thermoanaerobacter sp. and the like. More in particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacterium species no. 446 (Morinaga et al., 1990, J. Biotechnol., Vol. 14, p. 187-194), Acetobacterium wieringae (DSM 1911), Acetobacterium woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium ljungdahlii (DSM 13528), Clostridium ljungdahlii C-01 (ATCC 55988), (ATCC 55989), Clostridium ljungdahlii ERI-2 (ATCC 55380), Clostridium ljungdahlii O-52 Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004, Biotechnol. Let., Vol. 29, p. 1607-1612), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui).

More in particular, the acetogenic bacteria may be selected from the group consisting of Acetbacterium woodii, Alkalibaculum bacchi, Blautia producta, Clostridium aceticum, Clostridium autoethanogenum, Clostridium carboxidivorans, Clostridium drakei, Clostrdium formicoaceticum, Clostridium ljungdahlii, Clostridium magnum, Butyribacterium methyotrphoicum, Clostridium scatologenes, Eubacterium limosum, Moorella thermoacetica, Sporomusa ovate, Sporomusa silvacetica, Sporomusa sphaeroides, Oxobacter pfennigii, and Thermoanaerbacter kiuvi. More in particular, the acetogenic bacteria may be selected from the group consisting of Clostridium autoethanogenum and Clostridium ljungdahlii. Even more in particular, the acetogenic bacteria may be Clostridium autoethanogenum.

The term “fermentation” as used herein refers to a process for the production of one of more organic compounds by the anaerobic metabolism of the acetogenic bacterium in a medium suitable for the growth of the bacterium. This medium suitable for the growth of the bacterium refers to a fermentation medium comprising the ingredients necessary for the anaerobic bacterial growth and production of the alcohol. The medium will usually include carbon, nitrogen, phosphorus and sulphur sources, nutrients, trace elements, salts vitamins and so forth. Sulphur is usually an essential element in fermentation medium for the production of various organic compounds from a carbon source. The inventors have surprisingly found that when sulphur is present as a thiocarboxylate (salt) in the fermentation medium, a smaller amount of thiocarboxylate needs to be added to the medium compared to the other sulphur sources such as sulfide and cysteine which are usually used in conventional fermentation mediums as sources of sulphur. Thiocarboxylate makes relatively more sulphur bioavailable for consumption by the cells compared to the conventional sources of sulphur thus allowing the fermentation process to be more efficient and cost-effective.

The fermentation may be carried out under suitable conditions. The term “under suitable conditions” as used herein refers to the physical and chemical parameters of the fermentation medium necessary for the growth of the acetogenic bacteria and/or production of the desired organic compound, comprising pH, temperature, salinity, pressure, dissolved oxygen concentration, nitrogen requirements and substrate, nutrient and trace element concentrations and the like.

A skilled person would understand the suitable conditions necessary to carry out the method according to any aspect of the present invention. In particular, the conditions in the container (e.g. fermenter) may be varied depending on the acetogenic bacteria used. The varying of the conditions to be suitable for the optimal functioning of the microorganisms is within the knowledge of a skilled person.

In one example, the method according to any aspect of the present invention may be carried out in an aqueous medium with a pH between 5 and 8, 5.5 and 7. The pressure may be between 1 and 10 bar.

Acetogenic bacteria need to convert a carbon source to at least one organic compound. In particular, the cells are brought into contact with a carbon source which includes monosaccharides (such as glucose, galactose, fructose, xylose, arabinose, or xylulose), disaccharides (such as lactose or sucrose), oligosaccharides, and polysaccharides (such as starch or cellulose), one-carbon substrates and/or mixtures thereof. The carbon source used according to any aspect of the present invention may comprise carbon dioxide and/or carbon monoxide. A skilled person would understand that many possible sources for the provision of CO and/or CO₂ as a carbon source exist. It can be seen that in practice, as the carbon source according to any aspect of the present invention any gas or any gas mixture can be used which is able to supply the microorganisms with sufficient amounts of carbon, so that any organic compound may be formed from the source of CO and/or CO₂.

In one example, the carbon source comprises at least 50% by volume, at least 70% by volume, particularly at least 90% by volume of CO and/or CO₂, wherein the percentages by volume-% relate to all carbon sources that are available to the anerobic microorganism according to any aspect of the present invention. Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the microorganism according to any aspect of the present invention.

According to any aspect of the present invention, the carbon source may be synthesis gas. Synthesis gas can for example be produced as a by-product of coal gasification. Accordingly, the microorganism according to any aspect of the present invention may be capable of converting a substance which is a waste product into a valuable resource. In another example, synthesis gas may be a by-product of gasification of widely available, low-cost agricultural raw materials for use with the microorganism of the present invention to produce at least one organic compound.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Syngas may also be a product of electrolysis of CO₂. A skilled person would understand the suitable conditions to carry out electrolysis of CO₂ to produce syngas comprising CO in a desired amount.

Usually, a portion of the synthesis gas obtained from the gasification process is first processed in order to optimize product yields, and to avoid formation of tar. Cracking of the undesired tar and CO in the synthesis gas may be carried out using lime and/or dolomite. These processes are described in detail in for example, Reed, 1981.

An advantage of the present invention may be that much more favorable CO₂/CO mixtures of raw materials can be used. These various sources include natural gas, biogas, coal, oil, plant residues and the like. The overall efficiency, organic compound productivity and/or overall carbon capture of the method of the present invention may be dependent on the stoichiometry of the CO₂, CO, and H₂ in the continuous gas flow. The continuous gas flows applied may be of composition CO₂ and H₂. In particular, in the continuous gas flow, concentration range of CO₂ may be about 10-50%, in particular 3% by weight and H₂ would be within 44% to 84%, in particular, 64 to 66.04% by weight. In another example, the continuous gas flow can also comprise inert gases like N₂, up to a N₂ concentration of 50% by weight.

Generally, the carbon source comprises at least 50% by volume, at least 70% by volume, particularly at least 90% by volume of CO₂, wherein the percentages by volume-% relate to all carbon sources that are available to the acetogenic bacteria in the fermentation medium.

Mixtures of sources can be used as a carbon source.

According to any aspect of the present invention, a reducing agent, for example hydrogen may be supplied together with the carbon source. In particular, this hydrogen may be supplied when the C and/or CO₂ is supplied and/or used. In one example, the hydrogen gas is part of the synthesis gas present according to any aspect of the present invention. In another example, where the hydrogen gas in the synthesis gas is insufficient for the method of the present invention, additional hydrogen gas may be supplied.

The term “contacting”, as used herein, means bringing about direct contact between the acetogenic bacteria according to any aspect of the present invention and the carbon source. For example, the cell in the fermentation medium and the carbon source may be in different compartments. In particular, the carbon source may be in a gaseous state and added to the fermentation medium comprising the cells according to any aspect of the present invention.

According to any aspect of the present invention the organic compound may be at least one substituted and/or unsubstituted organic compound. The substituted or unsubstituted organic compound may be selected from the group consisting of acids, alcohols and/or diols. In particular, the organic compound may be selected from the group consisting of carboxylic acids, dicarboxylic acids, hydroxycarboxylic acids, carboxylic acid esters, hydroxycarboxylic acid esters, alcohols, aldehydes, ketones, amines, amino acids, and the like. In one example, the organic compounds according to any aspect of the present invention may be carboxylic acids, hydroxycarboxylic acids, carboxylic acid esters and/or alcohols. More in particular, these organic compounds comprise 1 to 36, 4 to 32, 6 to 20, or in particular 8 to 12 or 1 to 8 carbon atoms. Even more in particular, the alcohol is at least one C₁-C₈ alcohol and the acid is at least one C₁-C₈ acid. In particular, the organic compound may be selected from the group consisting of acetate, butyrate, propionate, caproate, ethanol, propanol, butanol, 2,3-butanediol, isopropanol, propylene, butadiene, isobutylene, ethylene, lactic acid, hexanoic acid and/or acetic acid. Even more in particular, the organic compound according to any aspect of the present invention may be lactic acid, acetic acid, hexanoic acid and/or ethanol.

The organic compounds produced according to any aspect of the present invention may be retrieved using any separation methods known in the art. For example, some of the organic compounds like ethanol may be recovered from the fermentation medium using fractional distillation or evaporation, and extractive fermentation. Extractive fermentation involves the use of a water-miscible solvent that presents a low toxicity risk to the anaerobic cell used in the fermentation process to recover the ethanol from the fermentation medium. Oleyl alcohol is a solvent that may be used in this type of extraction process.

In another example, an alkyl-phosphine oxide of general formula 1

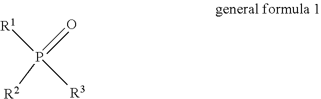

with R¹, R² and R³ selected from alkyl radicals containing 6 to 12, preferably 8 to 10, more preferably 8 or 10, carbon atoms, with the proviso, that at least two of R¹, R² and R³ differ from each other may be used to extract the organic compounds produced according to any aspect of the present invention. The alkyl-phosphine oxide may comprise at least two different alkyl radicals per alkyl-phosphine oxide molecule, for extracting the organic compound according to any aspect of the present invention.

According to yet another aspect of the present invention, there is provided a use of the fermentation medium according to any aspect of the present invention in a method for producing at least one alcohol and/or acid from a carbon source in the presence of hydrogen, wherein the carbon source comprises carbon monoxide and/or carbon dioxide.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Cultivation of Clostridium autoethanogenum with Potassium Thioacetate

The homoacetogenic bacterium Clostridium autoethanogenum was cultivated on synthesis gas in a mineral medium with potassium thioacetate as reduced sulphur source. All cultivation steps were carried out under anaerobic conditions in pressure-resistant glass bottles that can be closed airtight with a butyl rubber stopper.

The pre-cultivation of Clostridium autoethanogenum was carried out in a 1000 mL pressure-resistant glass bottle in 250 ml of EvoDM26 mineral medium (pH 6.2; 0.004 g/L Mg-acetate, 0.164 g/l Na-acetate, 0.016 g/L Ca-acetate, 0.025 g/l K-acetate, 0.107 mL/L H₃PO₄ (8.5%), 0.35 mg/L Co-acetate, 1.245 mg/L Ni-acetate×4H₂O, 20 μg/L d-biotin, 20 μg/L folic acid, 10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 2.109 mg/L (NH₄)₂Fe(SO₄)₂×6H₂O, 10.69 mg/L potassium thioacetate, acetic acid and NH₃ for pH regulation) at 37° C., 150 rpm and a ventilation rate of 1 L/h with a gas mixture of 62.5% H₂, 25% CO₂ and 12.5% CO in an open water bath shaker. It was inoculated with cells from a fresh culture of C. autoethanogenum to a start OD₆₀₀ₙₘ of 2.2 and cultivated for 200 h. The gas was discharged into the medium through surface aeration. The pH was hold at 6.2 by automatic addition of 4 M NH₃ solution. Fresh medium was continuously fed into the reactor and fermentation broth continuously removed from the reactor with a dilution rate of 1.8 d⁻¹.

For the main culture, as many cells from the preculture as necessary for an OD₆₀₀ₙₘ of 1.6 were transferred in 250 mL EvoDM26 medium. The chemolithoautotrophic cultivation was carried out in a 1 L pressure-resistant glass bottle at 37° C., 150 rpm and a ventilation rate of 1 L/h with a gas mixture of 62.5% H₂, 25% CO₂ and 12.5% CO in an open water bath shaker for 192 h. The gas was discharged into the medium through surface aeration. The pH was hold at 6.2 by automatic addition of 4 M NH₃ solution. Fresh medium was continuously fed into the reactor and fermentation broth continuously removed from the reactor with a dilution rate of 1.7 d⁻¹. During cultivation several 5 mL samples were taken to determinate OD₆₀₀ₙₘ, PH und product formation. The determination of the product concentrations was performed by semi-quantitative 1H-NMR spectroscopy. As an internal quantification standard sodium trimethylsilylpropionate (T(M)SP) was used.

During the main cultivation in EvoDM26 medium 2.048 g cell dry matter and 1.84 g acetate were produced. The sulphur concentration in the medium stayed at the same level about 3 mg/L over the whole cultivation time.

### Example 2

Formation of Acetic Acid and Ethanol from Synthesis Gas with Clostridium ljungdahlii and Refeeding of Cysteine

For the biotransformation of hydrogen and carbon dioxide to acetic acid and ethanol the homoacetogenic bacterium Clostridium ljungdahlii was cultivated on synthesis gas with refeeding of cysteine. All cultivation steps were carried out under anaerobic conditions in pressure-resistant glass bottles that can be closed airtight with a butyl rubber stopper.

For the preculture of C. ljungdahlii 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7H₂O; 0.02 g/L CaCl₂)×2H₂O; 20 mg/L nitrilotriacetic acid; 10 mg/L MnSO₄×H₂O; 8 mg/L (NH₄)₂Fe(SO₄)₂×6H₂O; 2 mg/L CoCl₂×6H₂O; 2 mg/L ZnSO₄×7H₂O; 0.2 mg/L CuCl₂×2H₂O; 0.2 mg/L Na₂MoO₄×2H₂O; 0.2 mg/L NiCl₂×6H₂O; 0.2 mg/L Na₂SeO₄; 0.2 mg/L Na₂WO₄×2H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B12; 50 μg/L p-aminobenzoate; 50 μg/L lipoic acid; approx. 67.5 mg/L NaOH) with additional 400 mg/L L-cysteine-hydrochloride and 400 mg/L Na₂S×9H₂O were inoculated with 5 mL of a frozen cryo stock. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 69 h till OD₆₀₀ₙₘ>0.4. The gas was discharged into the medium through a sparger with a pore size of 10 μm, which was mounted in the center of the reactors. Then the cell suspension was centrifuged and the cell pellet was resuspended in fresh CGF1 Medium.

For the production phase, as many washed cells from the preculture of C. ljungdahlii as necessary for an OD₆₀₀ₙₘ of 0.2 were added to 100 ml mineral medium (CGF1 medium, pH 6.5, 1.4 g/L KOH, 2 g/L (NH₄)₂SO₄, 1 g/L KH₂PO₄, 1 g/L K₂HPO₄, 10 mg/L FeSO₄×7H₂O, 3.8 mg/L MnSO₄×1H₂O, 246 mg/L MgSO₄×7H₂O, aerated for 30 min with a premixed gas with 67% H₂ and 33% CO₂). In the beginning culture A was supplemented with additional 400 mg/L L-cysteine-hydrochloride and cultures B and C with 200 mg/L L-cysteine-hydrochloride each. The cultivation was carried out in 500 mL pressure-resistant glass bottles at 37° C., 150 rpm in an open water bath shaker for 163 h which were aerated to an overpressure of 0.8 bar with a premixed gas with 67% H₂, 33% CO₂ one time a day. The pH was hold >5.0 by intermittent additions of a 140 g/L KOH solution. During the cultivation several 5 mL samples were taken to determinate OD₆₀₀ₙₘ, pH und product formation. Culture C was refed with 200 mg/l L-cysteine-hydrochloride after 18, 41, 65 and 89 h of cultivation. The determination of the product concentrations was performed by semiquantitative 1H-NMR spectroscopy. As an internal quantification standard sodium trimethylsilylpropionate (T (M) SP) was used.

During the production phase the concentrations of acetate and ethanol in culture C increased more than in culture A and B (see table 1), also culture C had a stronger cell growth than cultures A and B. In all three cultures all the added L-cysteine was consumed completely.

TABLE 1
Cell growth and product formation of cultures of Clostridium ljungdahlii
in CGF1 mineral medium on synthesis gas with 67% H₂ and 33% CO₂ with different
initial concentrations of L-cysteine and partial refeeding of L-cysteine.

| | Time | | Acetate | Ethanol | L-Cysteine | Feed [mg/L |
|---|---|---|---|---|---|---|
| Culture | [h] | OD(600 nm) | [mg/L] | [mg/L] | [mg/L] | Cys-HCl) |
| A | 0 | 0.206 | 34.0 | 2.0 | 270.0 | — |
| | 18.75 | 0.232 | 1400.0 | 60.0 | 70.0 | — |
| | 41.5 | 0.293 | 2550 | 420 | 0.0 | — |
| | 46.75 | 0.281 | 2700 | 480 | 0.0 | — |
| | 65.5 | 0.324 | 3500 | 580 | 0.0 | — |
| | 69.5 | 0.366 | 3700 | 650 | 0.0 | — |
| | 89.75 | 0.324 | 4350.0 | 580.0 | 0.0 | — |
| | 95.5 | — | — | — | 0.0 | — |
| | 163.5 | 0.339 | 6100.0 | 670.0 | 0.0 | — |
| B | 0 | 0.197 | 35.0 | 1.9 | 132.5 | — |
| | 18.75 | 0.261 | 1400.0 | 100.0 | 0.0 | — |
| | 41.5 | 0.279 | 2150 | 580 | 0.0 | — |
| | 46.75 | 0.269 | 2200 | 630 | 0.0 | — |
| | 65.5 | 0.271 | 2200 | 600 | 0.0 | — |
| | 69.5 | 0.259 | 2200 | 590 | 0.0 | — |
| | 89.75 | 0.241 | 2350.0 | 560.0 | 0.0 | — |
| | 95.5 | — | — | — | 0.0 | — |
| | 163.5 | 0.237 | 2550.0 | 570.0 | 0.0 | — |
| C | 0 | 0.209 | 27.0 | 2.0 | 120.0 | — |
| | 18.75 | 0.245 | 1400.0 | 85.0 | 0.0 | 200 |
| | 41.5 | 0.339 | 3000 | 510 | 0.0 | 200 |
| | 46.75 | 0.335 | 3250 | 580 | 123.0 | — |
| | 65.5 | 0.399 | 4250 | 790 | 0.0 | 200 |
| | 69.5 | 0.392 | 4400 | 860 | 108.0 | — |
| | 89.75 | 0.431 | 5000.0 | 810.0 | 0.0 | 200 |
| | 95.5 | 0.434 | 5600.0 | 880.0 | 89.0 | — |
| | 163.5 | 0.481 | 7700.0 | 960.0 | 0.0 | — |

### Example 3

Production of Ethanol by Clostridium autoethanogenum on Synthesis Gas with Boron

For the biotransformation of hydrogen, carbon monoxide and carbon dioxide to ethanol the homoacetogenic bacterium Clostridium autoethanogenum was cultivated on synthesis gas. All cultivation steps were carried out under anaerobic conditions in pressure-resistant glass bottles that can be closed airtight with a butyl rubber stopper.

For the preculture 400 ml medium (EvoDM01-medium: pH=5.8; 0.407 g/L MgCl₂*6H₂O, 0.117 g/L NaCl, 0.294 g/L CaCl₂)₂*2H₂O, 1.864 g/L KCl, 0.375 ml/L H₃PO₄, 19.8 mg/L FeCl₂×4H₂O, 0.5 g/L cysteine-HCl, 3.92 g/L NH₄-acetate, 0.396 mg/L MnCl₂×4H₂O, 0.476 mg/L CoCl₂×6H₂O, 0.682 mg/L ZnCl₂, 0.124 mg/L H₃BO₃, 0.484 mg/L Na₂MoO₄×2H₂O, 0.346 mg/L Na₂SeO₃*5 H₂O, 1.189 mg/L NiCl₂×6H₂O, 0.660 mg/L Na₂WO₄×2H₂O, 20 μg/L d-biotin, 20 μg/L folic acid, 10 μg/L pyridoxine-HCl, 50 μg/L thiamine-HCl×H₂O, 50 μg/L riboflavin, 50 g/L nicotinic acid, 50 μg/L Ca-pantothenate, 50 μg/L vitamin B12, 50 μg/L p-aminobenzoate, 50 μg/L lipoic acid) were inoculated with cells from a fresh culture of C. autoethanogenum to a start OD₆₀₀ₙₘ of 0.1. The chemolithoautotrophic cultivation was carried out in a 0.5 L pressure-resistant glass bottle at 37° C., 150 rpm and a ventilation rate of 2.3 L/h with a premixed gas with 60% H₂, 20% CO₂ and 20% CO in an open water bath shaker for 476 h. The gas was discharged into the medium through an aeration membrane, which was mounted in the center of the reactors. The pH was hold at 5.5 by automatic addition of 2.5 M NH₃ solution. Fresh medium was continuously fed to the reactor and fermentation broth continuously removed from the reactor with a dilution rate of 1.0 d⁻¹.

After the pre-cultivation, the cell suspension was centrifuged (10 min, 4200 rpm) and the pellet was resuspended in fresh main culture medium. For the main culture, as many cells from the preculture as necessary for an OD₆₀₀ₙₘ of 1.0 were transferred in 400 mL medium. For the main culture also EvoDM01 mineral medium was used. The chemolithoautotrophic cultivation was carried out in a 0.5 L pressure-resistant glass bottle at 37° C., 150 rpm and a ventilation rate of 2.3 L/h with a premixed gas with 60% H₂, 20% CO₂ and 20% CO in an open water bath shaker for 45 h. The gas was discharged into the medium through an aeration membrane, which was mounted in the center of the reactors. The pH was hold at 5.5 by automatic addition of 2.5 M NH₃ solution. Fresh medium was continuously fed to the reactor and fermentation broth continuously removed from the reactor with a dilution rate of 1.0 d⁻¹. During cultivation several 5 mL samples were taken to determinate OD₆₀₀ₙₘ, pH und product formation. The determination of the product concentrations was performed by semiquantitative 1H-NMR spectroscopy. As an internal quantification standard sodium trimethylsilylpropionate (T (M) SP) was used.

During the main cultivation in EvoDM01 medium 3.22 g ethanol and 1.11 g acetate were produced.

### Example 4

Cultivation of Clostridium autoethanogenum with Low Potassium Thioacetate Concentration

The homoacetogenic bacterium Clostridium autoethanogenum was cultivated together with the chain elongating bacterium Clostridium kluyveri in a co-culture on synthesis gas in a mineral medium with potassium thioacetate as reduced sulfur source. The cultivation was carried out under anaerobic conditions in a pressure-resistant stainless steel bubble column loop reactor.

The cultivation was run at 37° C. and an overpressure of 2 bar as a continuous fermentation with continuous feeding of 300 L/h of a mixture of water, substrates, salts, trace elements and vitamins. The pH was automatically hold at 5.80 with ammonia feeding. The outlet stream of 300 L/h out of the fermenter was for 98.2% as permeate with cell retention and for 1.8% as purge without cell retention. The gas was discharged into the medium through a sparger with a ventilation rate of ˜ 1000 L/h as a gas mixture of 62.5% H₂ and 37.5% CO₂.

The media feed consisted of 0.004 g/L Mg-acetate×4H₂O, 0.164 g/L Na-acetate, 0.016 g/L Ca-acetate, 0.245 g/l K-acetate, 0.107 mL/L H₃PO₄ (8.5%), 0.35 mg/L Co-acetate, 1.245 mg/L Ni-acetate×4H₂O, 20 μg/L d-biotin, 20 μg/L folic acid, 10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 2.109 mg/L (NH₄)₂Fe(SO₄)₂×6H₂O, 10.69 mg/L potassium thioacetate, 6.73 g/L ethanol and NH₃ for pH regulation.

The culture was previously inoculated with cells from fresh cultures of C. autoethanogenum and C. kluyveri and was already running since >10.000 h full continuously as stable coculture at an optical density (OD₆₀₀ₙₘ) of ˜ 9.0. Fresh medium was continuously fed into the reactor and fermentation broth continuously removed from the reactor with a dilution rate of 2.8 d⁻¹. During cultivation several 5 mL samples were taken to determinate OD₆₀₀ₙₘ, PH und product formation. The determination of the product concentrations was performed by semiquantitative 1H-NMR spectroscopy. As an internal quantification standard sodium trimethylsilylpropionate (T (M) SP) was used.

The steady state concentration of the educts and products in the reactor were about 3.27 g/L ethanol, 0.90 g/L acetate, 0.91 g/L butyrate and 3.61 g/L hexanoate. 50h after decreasing the potassium thioacetate concentration in the media feed to 10% (1.69 mg/L), the steady state concentrations decreased to 0.50 g/L acetate, 0.53 g/L butyrate and 2.41 g/L hexanoate, the ethanol concentration raised to 4.37 g/L. The OD₆₀₀ₙₘ decreased from 9.0 to 7.60 and the CO₂ turnover decreased from 70% to 50% during this time.

### Example 5

Cultivation of Clostridium autoethanogenum with High Potassium Thioacetate Concentration

The homoacetogenic bacterium Clostridium autoethanogenum was cultivated together with the chain elongating bacterium Clostridium kluyveri in a co-culture on synthesis gas in a mineral medium with potassium thioacetate as reduced sulfur source. The cultivation was carried out under anaerobic conditions in a pressure-resistant stainless steel bubble column loop reactor.

The cultivation was run at 37° C. and an overpressure of 2 bar as a continuous fermentation with continuous feeding of 300 L/h of a mixture of water, substrates, salts, trace elements and vitamins. The pH was automatically hold at 5.80 with ammonia feeding. The outlet stream of 300 L/h out of the fermenter was for 98.2% as permeate with cell retention and for 1.8% as purge without cell retention. The gas was discharged into the medium through a sparger with a ventilation rate of ˜ 1000 L/h as a gas mixture of 62.5% H₂ and 37.5% CO₂.

The media feed consisted of 0.004 g/L Mg-acetate×4H₂O, 0.164 g/L Na-acetate, 0.016 g/L Ca-acetate, 0.245 g/l K-acetate, 0.107 mL/L H₃PO₄ (8.5%), 0.35 mg/L Co-acetate, 1.245 mg/L Ni-acetate×4H₂O, 20 μg/L d-biotin, 20 μg/L folic acid, 10 μg/L pyridoxine-HCl, 50 μg/L thiamine-HCl, 50 μg/L Riboflavin, 50 μg/L nicotinic acid, 50 μg/L Ca-pantothenate, 50 μg/L Vitamin B12, 50 μg/L p-aminobenzoate, 50 μg/L lipoic acid, 2.109 mg/L (NH₄)₂Fe(SO₄)₂×6H₂O, 10.69 mg/L potassium thioacetate, 6.73 g/L ethanol and NH₃ for pH regulation.

The culture was previously inoculated with cells from fresh cultures of C. autoethanogenum and C. kluyveri and was already running since >13.000 h full continuously as stable coculture at an optical density (OD₆₀₀ₙₘ) of ˜ 11.8. Fresh medium was continuously fed into the reactor and fermentation broth continuously removed from the reactor with a dilution rate of 2.8 d⁻¹. During cultivation several 5 mL samples were taken to determinate OD₆₀₀ₙₘ, PH und product formation. The determination of the product concentrations was performed by semiquantitative 1H-NMR spectroscopy. As an internal quantification standard sodium trimethylsilylpropionate (T (M) SP) was used.

The steady state concentration of the educts and products in the reactor were about 2.67 g/L ethanol, 1.10 g/L acetate, 1.08 g/L butyrate and 3.75 g/L hexanoate. 50 h, 100 h and 150 h after increasing the potassium thioacetate concentration in the media feed to 300% (32.07 mg/L), the steady state concentrations of the educts and products stayed on the same level. Also, the OD₆₀₀ₙₘ stayed at 11.80 and the CO₂ turnover stayed at 70% during this time.

1. An aqueous fermentation medium comprising sulphur in the form of at least one thiocarboxylate, wherein the thiocarboxylate has a chemical structure of Formula I: 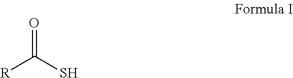
and R═H, alkyl, COOH, COSH, and wherein the alkyl groups may also contain OH, COSH and/or COOH and wherein the concentration of the thiocarboxylate is 2 to 20 mg/L in the fermentation medium.

2. The fermentation medium according to claim 1, wherein the thiocarboxylate is selected from the group consisting of thioacetate, thioformate, thiobutyrate, thiolactate, thiopropionate, thiohexanoate, thiooctanoate, thiodecanoate, thiododecanoate, and thiocitrate salt.

3. The fermentation medium according to claim 1, wherein the thiocarboxylate is selected from the group consisting of thioacetate, thiobutyrate and thiocitrate.

4. The fermentation medium according to claim 1, wherein the medium further comprises an iron, nickel and/or cobalt salt.

5. A method of producing at least one organic compound from a carbon source, the method comprising:
contacting at least one anaerobic bacteria to the carbon source in a fermentation medium, wherein the fermentation medium comprises sulphur in the form of at least one thiocarboxylate and wherein the thiocarboxylate has a chemical structure of Formula I: 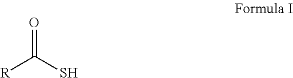
and R═H, alkyl, COOH, COSH, and wherein the alkyl groups may also contain OH, COSH and/or COOH and wherein the concentration of the thiocarboxylate is 2 to 20 mg/L in the fermentation medium.

6. The method according to claim 5, wherein the anaerobic bacteria is an acetogenic cell and the acetogenic cell is preferably selected from the group consisting of Clostridium autothenogenum DSMZ 19630, Clostridium ragsdahlei ATCC no. BAA-622, Clostridium autoethanogenum, Moorella sp HUC22-1, Moorella thermoaceticum, Moorella thermoautotrophica, Rumicoccus productus, Acetoanaerobum, Oxobacter pfennigii, Methanosarcina barkeri, Methanosarcina acetivorans, Carboxydothermus, Desulfotomaculum kutznetsovii, Pyrococcus, Peptostreptococcus, Butyribacterium methylotrophicum ATCC 33266, Clostridium formicoaceticum, Clostridium butyricum, Lactobacillus delbrukii, Propionibacterium acidoproprionici, Proprionispera arboris, Anaerobierspirillum succiniproducens, Bacterioides amylophilus, Becterioides ruminicola, Thermoanaerobacter kivui, Acetobacterium woodii, Acetoanaerobium notera, Clostridium aceticum, Butyribacterium methylotrophicum, Moorella thermoacetica, Eubacterium limosum, Peptostreptococcus productus, Clostridium ljungdahlii, Clostridium ATCC 29797 and Clostridium carboxidivorans.

7. The method according to claim 5, wherein the thiocarboxylate is selected from the group consisting of thioacetate, thioformate, thiobutyrate, thiolactate, thiopropionate, thiohexanoate, thiooctanoate, thiodecanoate, thiododecanoate, and thiocitrate salt.

8. The method according to claim 5, wherein the thiocarboxylate is selected from the group consisting of thioacetate, thiobutyrate and thiocitrate.

9. The method according to claim 5, wherein the medium further comprises an iron, nickel and/or cobalt salt.

10. The method according to claim 5, wherein the carbon source is in the presence of hydrogen, and the carbon source comprises carbon monoxide and/or carbon dioxide.

11. The method according to claim 5, wherein the organic compound is at least one alcohol and/or acid.

12. The method according to claim 11, wherein the alcohol is at least one C₁-C₈ alcohol and the acid is at least one C₁-C₈ acid.

13. Use of the fermentation medium according to claim 1 in a method for producing at least one alcohol and/or acid from a carbon source in the presence of hydrogen, wherein the carbon source comprises carbon monoxide and/or carbon dioxide.
